(19) United States
(12) Patent Application Publication
(10) Pub. No.: US 2022/0175825 A1
(43) Pub. Date: Jun. 9, 2022

(54) COMPOSITION AND ASSOCIATED DELIVERY DEVICE FOR HYDROGEN THERAPY

(71) Applicants: UNIVERSITE GRENOBLE ALPES, Saint Martin D'Heres (FR); INSTITUT POLYTECHNIQUE DE GRENOBLE, Grenoble (FR); CENTRE NATIONAL DE LA RECHERCHE SCIENTIFIQUE, Paris (FR); CENTRE HOSPITALIER UNIVERSITAIRE GRENOBLE ALPES, La Tronche (FR)

(72) Inventors: Jean-Pierre ALCARAZ, Pontcharra (FR); Donald MARTIN, Gieres (FR); Philippe CINQUIN, Saint Nazaire Les Eymes (FR)

(21) Appl. No.: 17/437,361

(22) PCT Filed: Mar. 4, 2020
(86) PCT No.: PCT/EP2020/055735

§ 371 Date: Sep. 8, 2021

(30) Foreign Application Priority Data
Mar. 8, 2019 (FR) 1902392

(51) Int. Cl. A61K 33/00; A61K 33/06

(52) U.S. Cl. CPC A61K 33/00; A61K 33/06

(57) ABSTRACT

Hydrogen therapy and more particularly a composition having: hydride able and intended to dissolve on contact with an aqueous medium and therefore to release dihydrogen, and a formulation agent of the hydride. The formulation agent configured to bring the hydride into contact with an environment of the composition in at least one physiological condition observable in a human or non-human animal body. Thus, by degradation of the formulation agent and dissolution of the hydride, the composition allows releasing dihydrogen in a dissolved form in a targeted portion of the human or non-human animal body.

### TECHNICAL FIELD

The invention relates to the field of hydrogen therapy. It finds a particularly advantageous application in the treatment of numerous pathologies, including Alzheimer's and Parkinson's diseases.

### STATE OF THE ART

Hydrogen therapy has an increasing interest for treating a large number of pathogens related to an oxidative stress. A recent publication (Ichihara, M., Sobue, S., Ito, M., Ito, M., Hirayama, M., & Ohno, K. (2015), “Beneficial biological effects and the underlying mechanisms of molecular hydrogen-comprehensive review of 321 original articles”, Medical gas research, 5(1), 12) of more than 300 articles lists no less than 166 pathologies where hydrogen has been tested for its anti-oxidative virtues. For example, a study in mice and a clinical test in humans on 73 patients suffering from Alzheimer's disease demonstrates the effectiveness of the ingestion of 300 mL a day of hydrogen water not only to reduce the loss of memory in patients but also to increase their life expectancy (Nishimaki, K., Asada, T., Ohsawa, I., Nakajima, E., Ikejima, C., Yokota, T., . . . & Ohta, S. (2017), “Effects of molecular hydrogen assessed by an animal model and a randomised clinical study on mild cognitive impairment”, Current Alzheimer research).

Current techniques for administering this molecular hydrogen are:
In the form of a gas that could be inhaled formed by electrolysis (Camara R, Huang L, Zhang JH (2016), “The production of high dose hydrogen gas by the AMS-H-01 for treatment of disease”, Med Gas Res, 6(3):164-166). This technique is expensive and inconvenient, as it requires a ventilator-type apparatus and an electric power supply;
In the form of hydrogen water produced by hydrolysis. This method is known from the invention of Volta's cell. In the medical field, mobile hydrolysers, such as those described in the patent documents US 20130043124 A1 and EP 2567942 B1, are commercialised for the production of therapeutic hydrogen. The saturation limit concentration in water is 1.57 ppm. The amount of dihydrogen delivered by this method is limited by the saturation of water with dihydrogen. For example, they are from 0.8 to 1.3 ppm for the “SPE” system of AlkaVoda, depending on the hardness of the used water. A hydrolyser requires an apparatus having an electric power supply. Furthermore, dioxygen is also produced, which limits the amount of dissolved hydrogen. Some hydrolysers separate the emitted gases, namely oxygen and hydrogen, so as to keep only hydrogen in a dissolved form;
In the form of hydrogen water produced from hydrides packed in a pouch permeable to gases (palladium paper type). This system not only allows for the absence of dioxygen production, but also for an oversaturation of the molecular hydrogen (about 5 to 8 ppm). Such a solution is commercialised under the name “trust 8.0” or “Hydra” or Hfactor™. Nonetheless, the resulting powders are indigestible, the half-life of the dissolved dihydrogen is very short which justifies the indication “drink the hydrogen water as quickly as possible to benefit from a sufficient concentration”;
In the form of hydrogen water produced from hydrides. Such a solution is commercialised under the name AquaH2™. Herein again, the molecular hydrogen is released by adding water extemporaneously. A drawback of this technique is that, as the dissolution of the hydrogen depends on the hardness of the water, variations of the calcium concentration in drinking water could vary the concentration of dissolved dihydrogen;
Similarly, the delivery of hydrogen water generates a discontinuity in the administration of the treatment: the dihydrogen concentration is therefore subjected to wide variations.

Hence, current techniques have limitations. In particular, none of them allows delivering an accurate dose of dihydrogen at a targeted location of the human or animal body.

Hence, an object of the present invention is to provide a new composition that allows overcoming at least part of the drawbacks of the techniques known until now.

More particularly, an object of the present invention is to provide a new composition that allows delivering an accurate dose of dihydrogen at a targeted location of the human or animal body and/or over a controlled period of time, possibly longer than the delivery durations reached via the administration methods according to the prior art.

The other objects, features and advantages of the present invention will appear upon examining the following description and the appended drawings. It should be understood that other advantages could be incorporated.

### SUMMARY

To achieve this objective, according to one embodiment, the present invention provides a composition comprising:
at least one hydride able and intended to dissolve on contact with an aqueous medium and therefore to release dihydrogen in a dissolved form, and
at least one formulation agent of said at least one hydride, the formulation agent being configured so as to be degraded in at least one physiological condition observable in the human or animal body, so as to release said at least one hydride.

According to a combinable or alternative embodiment, the composition comprises:
at least one hydride able and intended to dissolve on contact with an aqueous medium and therefore to release dihydrogen in a dissolved form, and
at least one formulation agent of said at least one hydride, the formulation agent being configured so as to set the at least one hydride in contact with an environment of the composition in at least one physiological condition observable in the human or animal body.

According to a combinable or alternative embodiment, the composition comprises:
at least one hydride able and intended to dissolve on contact with an aqueous medium and therefore to release dihydrogen in a dissolved form, and
at least one formulation agent of said at least one hydride, the formulation agent being configured so as to isolate the hydride from an environment of the composition.

By “formulation”, it should be understood the determination of the relative amounts of various elements included in a composition, and possibly the determination of the relative arrangement of these various elements with respect to one another. A formulation agent actively contributes to this determination, at least as an element of the composition, and possibly as a structuring element of the composition.

By “physiological condition observable in the human or animal body”, it should be understood a condition defined by at least one physiological parameter such as the presence of water, the temperature, the pH, the concentration of mineral salts, etc. that could be observed at the level of at least one location of the human or animal body.

The non-degraded formulation agent is configured so as to isolate the hydride from an environment of the composition.

Moreover, the formulation agent could, more particularly, be configured so as to be degraded under at least one physiological condition observable in the human or animal body. By “degradation”, it should be understood that the protective properties of the formulation agent are modified under at least one physiological condition observable in the human or animal body. According to one possibility, the formulation agent could be configured so as to release the hydride only when the composition is under at least one specific physiological condition.

Thus, the degradation of said at least one formulation agent allows setting the hydride in contact with said aqueous medium and, consequently, the release of dihydrogen.

According to one possibility, the formulation agent is semi-permeable and allows setting the hydride in contact with the environment of the composition under at least one physiological condition observable in the human or animal body. According to one possibility, the formulation agent is semi-permeable to ions, to water and to at least one gas, in particular dihydrogen.

According to one possibility, by degradation of the formulation agent and dissolution of the hydride, the composition allows releasing dihydrogen in at least one targeted portion of the human or animal body, this targeted portion being potentially defined by said at least one physiological condition.

Henceforth, the released hydrogen molecules could be absorbed, assimilated or used by the organism, and more particularly by at least one target member or tissue.

Optionally, the composition according to the invention may further have at least any one of the following features:
On the one hand, the hydride and products formed by dissolution thereof and, on the other hand, the formulation agent and products formed by degradation thereof are pharmaceutically acceptable. Indeed, products derived from the dissolution of the hydride or from the degradation of the formulation agent could be formed which are biocompatible and could be eliminated with the excrements or with the fluids of the organism. The pharmaceutical acceptability, or the non-toxicity, of the hydride and of the formulation agent, as well as their by-products, is to be assessed, in particular in terms of limit dose, with regards to the disease that shall be treated, its proven or potential consequences, and the benefit that the human or animal subject might obtain from the targeted delivery of dihydrogen thanks to the composition according to the invention;
The formulation agent comprises at least one amongst:
   a container configured to contain the hydride,
   a coating configured to coat the hydride or particles of the hydride, and
   a binder for bonding together particles of the hydride;
the hydride is porous. Thus, the hydride offers an enlarged surface of contact with the aqueous medium for a more effective release of dihydrogen;
the hydride is in the form of particles whose particles preferably have an average size comprised between 10 nm and 10 μm. Thus, the hydride offers an enlarged surface of contact with the aqueous medium for a more effective release of dihydrogen. Furthermore, the hydride is thus easy to contain and/or bond;
the hydride is selected amongst: a silicon hydride, a magnesium hydride and a calcium hydride;
the hydride is based on a porous silicon, preferably non-passivated. The porosities could have a mesoscopic and/or nanoscopic size. The composition according to this last feature forms a preferred embodiment of the invention. It potentially combines the aforementioned advantages and is compatible with the following features of the composition;
the formulation agent is based on at least one amongst:
   a gastro-resistant material,
   a material soluble on contact with a medium with a determined potential of hydrogen (pH),
   a material based on a biodegradable polymer, such as the polylactic acid (PLA),
   a material degradable by external stimulation, such as an ultrasound stimulation,
   a material soluble on contact with an aqueous medium, and
   a gel
the formulation agent is based on a material selected so as to be degraded under at least one specific external stimulus. Thus, the composition according to either one of these last two features allows for a wide variety of modes, locations and flow rates of delivery in the human or animal body;
the formulation agent is based on a material selected so as to be degraded under at least one specific external stimulus, possibly independently of said at least one physiological condition. The composition according to this additional feature allows finely controlling the flow rate of delivery of dihydrogen in the organism; and
the composition may comprise a plurality of formulation agents configured together, for example in successive concentric layers or in superimposed planar layers, so as to be degraded either differently under said at least one physiological condition, or under physiological conditions that are different from each other, in particular so as to be degraded either differently under said at least one physiological condition, or to be degraded under different physiological conditions,
the composition could be free of any active substance in addition to the at least one hydride,
the composition may comprise a mixture of hydrides, and more particularly a mixture of hydrides comprising a silicon hydride and at least one other hydride, in particular an ion hydride such as calcium hydride or magnesium hydride,
the composition may comprise a mixture of hydrides, comprising a silicon hydride and at least one other hydride, the proportion of silicon hydride being higher than 20 weight %, and possibly higher than 50 weight %, and possibly higher than 75 weight % with respect to the total mass of hydride.

According to an embodiment that could have any one of the features hereinabove, the composition may further comprise at least one detection agent configured so as to allow detecting the ingestion of the composition and consequently allow measuring the therapeutic observance. The detection agent may be mixed with a therapeutic-sue hydride as described before. Alternatively or complementarily, the detection agent may be contiguous, structurally in the composition, to the therapeutic-use hydride as described before, the formulation agent as described before or a complementary formulation agent could be configured to this end. Preferably, the detection agent comprises at least one hydride able and intended to dissolve on contact with an aqueous medium in the human or animal body by releasing dihydrogen.

Another aspect of the present invention relates to a composition for its use as a medicine, the composition being as described hereinabove.

Another aspect of the present invention relates to a composition for its use in the treatment of at least one cardiovascular disease, such as the myocardial infarction, or in the treatment of at least one neurodegenerative disease, such as Parkinson's disease and Alzheimer's disease, the composition being as described hereinabove.

Another aspect of the present invention relates to a device for targeted delivery of dihydrogen in the human or animal body, the device comprising a composition as described hereinabove.

Optionally, the invention according to these different aspects may further have at least any one of the following features:
it could be formulated so as to be adapted to at least one administration route selected amongst an oral, parenteral, rectal, vaginal, ophthalmic, cutaneous, transdermal and respiratory administration; and
it could be formulated so as to be administered in one of the following forms: a pill, a capsule, a plaster, a contact lens (an ocular orthosis made of glass or of a plastic material, transparent, moulded so as to fit directly onto the eye, where it could correct the refraction thereof), and an implant (1E).

Advantageously, the device and the composition according to these last features could take on numerous forms corresponding to so many modes of administration of the composition.

Other aspects of the present invention relate to methods implementing a composition as described hereinabove.

The first one of these methods consists of a method for targeted delivery of dihydrogen in the human or animal body, from outside the human or animal body respectively, the method implementing a composition as described hereinabove, said composition being comprised in a targeted delivery device in one of the following forms: a pill, a capsule, a plaster and a contact lens.

The second method consists of a method for administering a substance beneficial to a human or animal body comprising the ingestion of a composition as described hereinabove.

A third method consists of a method for administering a liquid or a paste passing through a needle or a catheter and containing a composition as described hereinabove by injection, for example in an infarcted area or a tumour.

A fourth method consists of a method for administering by implantation an implantable medical device, such as a stent, a pacemaker (in particular a leadless pacemaker), or stimulation electrodes, coated in a composition as described hereinabove, allowing a local delivery of a dose of hydrogen aiming to reduce the inflammation related to the implantation of said device.

### BRIEF DESCRIPTION OF THE FIGURES

The aims, objects, as well as the features and advantages of the invention will appear better from the detailed description of an embodiment of the latter which is illustrated by the following appended drawings wherein:

FIGS. 1A to 1E illustrate sectional views of different embodiments of the composition and/or of the device according to the invention;

FIG. 2 illustrates a transparent view of some portions of a human body;

FIG. 3 schematically illustrates the adaptation capacity offered by the present invention to ensure a targeted delivery of dihydrogen; and

FIG. 4 illustrates a sectional view of an embodiment of the composition and/or of the device according to the invention comprising a detection agent.

FIG. 5A is a graph of the kinetics of release of dihydrogen induced by the dissolution of a composition comprising a magnesium hydride in a solution, according to the pH of the solution, according to different embodiments of the composition. FIG. 5B is a detailed graph of the initial time interval of FIG. 5A.

FIG. 6A is a graph of the kinetics of release of dihydrogen induced by the dissolution of a composition comprising a silicon hydride in a solution, according to the pH of the solution, according to different embodiments of the composition. FIG. 6B is a detailed graph of the initial time interval of FIG. 6A.

FIG. 7 is a graph of the kinetics of release of dihydrogen induced by the dissolution of different embodiments of the composition comprising a mixture of magnesium hydride and silicon hydride.

FIG. 8 is a graph of the kinetics of release of dihydrogen induced by the dissolution of a composition comprising a silicon hydride in a gel, according to an embodiment of the composition.

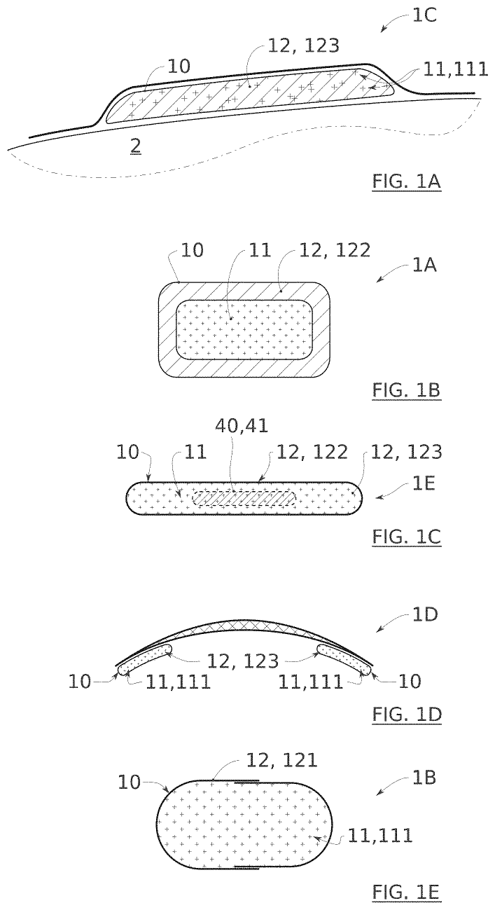

The drawings are provided as examples and do not limit the invention. They consist of schematic block diagrams intended to facilitate understanding of the invention and are not necessarily to the scale of the practical applications. In particular, the respective sizes of the different embodiments illustrated in FIGS. 1A to 1E are not intended to be compared to each other.

Expressions such as “equal, lower, higher” should be understood as comparisons that could allow for some tolerances, in particular depending on the scale of magnitude of the compared values and the measurement uncertainties. Substantially equal, lower or higher values fall within the scope of the invention.

The term “hydride” refers to a chemical compound consisting of hydrogen and at least one other chemical element that is less electronegative, or with a comparable electronegativity. More particularly, the hydrogen element in a hydrogen is in a reduced state.

By a parameter “substantially equal to/higher than/lower than” a given value, it should be understood that this parameter is equal to/higher than/lower than the given value, more or less 10%, and possibly more or less 5%, of this value.

The invention consists in providing an alternative to the ingestion of hydrogen water with the same, and possibly with more, therapeutic objectives and benefits.

Referring to FIGS. 1A to 1E, the invention primarily relates to a composition 10 comprising at least one hydride 11 and at least one formulation agent 12.

In particular, the hydride 11 is able and intended to dissolve on contact with an aqueous medium and therefore to release dihydrogen.

The formulation agent 12 allows formulating the composition 10. It formulates it at least by determining the relative amounts of the various elements included in the composition 10, and possibly also by determining the relative arrangement of these various elements with respect to one another. The formulation agent 12 actively participates to this determination, at least as an element of the composition 10, but also, where appropriate, as a structuring element of the composition 10. Indeed, the formulation agent 12 according to the invention could consist of a container 121, such as a capsule, as well as of a coating 122 to coat the hydride 11 or particles 111 of the hydride. Alternatively or complementarily, the formulation agent 12 according to the invention could also consist of a binder 123 for bonding particles 111 of the hydride together.

In the case where the formulation agent 12 consists of a container 121, the location of the human or animal body, and in particular the location of the gastrointestinal tract where the container 121 will be degraded, could be controlled through the selection of the composition and the thickness of the container 121, so as to have a targeted release of the molecular hydrogen. In this embodiment, the flow rate with which the molecular hydrogen will be released depends more on the form in which the hydride 11 is contained in the container 121. Of course, the joint use of a container 121 and of at least one amongst a coating 122 and a binder 123 is not excluded.

In the case where the formulation agent 12 consists of a coating 122 or a binder 123, the location of the human or animal body, and in particular the location of the gastrointestinal tract where the coating 122 or the binder 123 will be degraded, could be controlled through the selection of the composition and the thickness of the container 121. Thus, a targeted release of the molecular hydrogen is obtained, but above all, it is possible to obtain a prolonged release of hydrogen over the route of the composition 10 in the human or animal body 2, and in particular on at least one portion of the gastrointestinal tract, for example from the mouth 22 to the stomach 25 or from the duodenum 26 to the colon 30.

The non-degraded formulation agent 12, or before degradation thereof, is configured so as to isolate the hydride from the environment of the composition 10 and/or to keep the hydride in a determined form. Thus, except in the case of degradation, the formulation agent 12 allows preserving the hydride 11 contained in the composition 10 from any contact with the environment, and in particular from any contact with an environment that could cause the release of dihydrogen, in particular a possible surrounding aqueous medium.

More particularly, the formulation agent 12 is configured so as to be degraded in at least one physiological condition observable in the human or animal body. Its degradation allows releasing the hydride 11. Bearing in mind that the physiological conditions observable in the human or animal body vary from one member or tissue to another, it clearly arises that the administration of dihydrogen thanks to the composition according to the invention is closely linked to the physiological conditions that the target member or tissue has in a known manner.

Still more particularly, the formulation agent 12 could be configured so as to be degraded under at least one physiological condition observable in the human or animal body. More particularly, the formulation agent 12 could be configured so as to release the hydride only when the composition is under at least one specific physiological condition. Indeed, the formulation agent could be selected so as to be degraded when put in a specific surrounding condition defined by a physiologic parameter or a combination of physiologic parameters amongst which the presence, and possibly the amount, of water, the temperature, the pH, the concentration of mineral salts, etc., such a physiologic parameter or such a combination of physiologic parameters being observable at the level of at least one location, and possibly at a unique location, of the human or animal body.

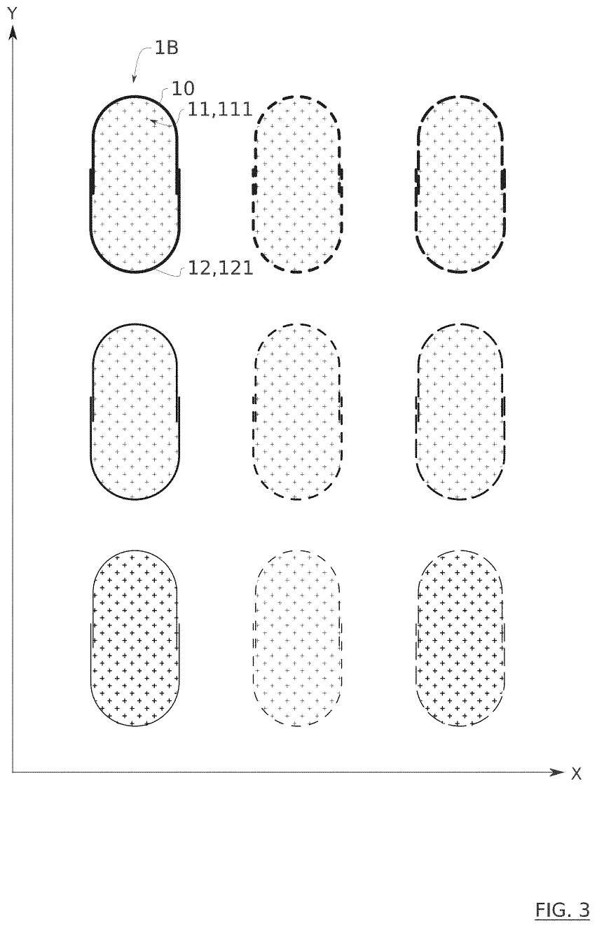

It is the formulation offered by the formulation agent 12 that actually allows controlling, following the intake of the medicine formed by the composition 10 and according to its mode of administration, at least one amongst the tome at which the delivery of dihydrogen will take place and the location where this delivery will take place. Given the foregoing, and this will be set out later on with reference to FIG. 3, it should be understood that this control is to be determined by a person skilled in the art wishing to deliver dihydrogen to a specific target member(s) or tissue(s) to treat a specific disease(s) thanks to the composition 10 according to the invention. For example, the company Evonik is supposed to have the required competencies for an accurate definition of the formulation to be adopted to meet a requirement set relating for example to at least one amongst the treatment of a determined specific disease, a determined administration route and a determined subject. As a guideline, it could be retained that the relationship between the formulation of the composition 10 and each type of pathology to be treated preferably depends at least on one amongst a targeted administration and an administration in the gastrointestinal tract, the latter could be targeted or expand over the tract. In the case of an expanded administration, it is interesting to formulate the composition 10 so as to have a prolonged release of molecular hydrogen along at least one portion of the tract, for a long-term benefit of at least one corresponding portion of the human or animal body.

Thus, the degradation of the formulation agent 12 allows setting the hydride 11 in contact with the aqueous medium that forms the location of the human or animal body where a specific physiological condition would prevail. Indeed, it could be considered that the specific physiological condition or a set of specific physiological conditions defines a member or a tissue in a differentiated manner with respect to the other members and tissues of the human or animal body, in particular with regards to the mode of administration of the composition 10. Consequently, thanks to the composition 10 according to the invention, the dihydrogen release could be completed at the level of at least one specific location of the human or animal body.

Complementarily or alternatively, the formulation agent 12 may be based on a material selected so as to be degraded under at least one specific external stimulus. For example, the degradation of the formulation agent 12 and therefore the delivery of dihydrogen could be ensured by activation of an external energy source, for example an ultrasound source, known to act on the formulation agent 12 by degrading it. Possibly, the external energy source may act on the formulation agent 12 through members and tissues other than the target member or tissue. Depending on the nature of the external energy, it could thus be considered to subject at least the target member or tissue to said specific external stimulus, at least when the composition 10 is present or applied therein. Thus, this possibility could allow releasing, or possibly annihilating, the constraint implied by the need for this agent to be degraded under a specific physiological condition on the selection of the formulation agent 12. The formulation agent 12 may then be selected only with the constraint that it is not degraded before the composition has reached, or has been applied on, the target member or tissue. To this end, the composition 10 according to the invention could be specifically conditioned, for example by a packaging or by its arrangement in a device for delivering the composition 10.

Alternatively, the degradation of the formulation agent 12, and therefore the delivery of dihydrogen, could be ensured by activation of an external electric current source, known to act on the formulation agent 12 by degrading it. In this case, the electric current may be supplied by an electric current generator connected to an electrode implanted in the patient, for example.

In another example, the degradation of the formulation agent 12 and therefore the delivery of dihydrogen could be ensured by activation of an internal energy source, for example an electric current source, known to act on the formulation agent 12 by degrading it. Where appropriate, the electric current could be supplied by a pacemaker, the delivery being controlled for example by a predetermined setpoint according to a physiological signal measured by the pacemaker or communicated to the pacemaker from outside the body of the patient.

Whether the formulation agent 12 is degradable in/under a specific physiological condition or under a specific external stimulus, it could be based on at least one amongst:
a gastro-resistant material,
a material soluble on contact with a medium with a determined potential of hydrogen (pH),
a material based on a biodegradable polymer, such as the polylactic acid (PLA),
a material degradable by external stimulation, such as an ultrasound stimulation,
a material soluble on contact with an aqueous medium, and
a gel.

These different materials may be degraded under different environmental conditions. They may also have different degradation rates, possibly in equivalent environmental conditions. For example, it is advantageous that the formulation agent 12 is based on a material selected so as to have a determined degradation rate in or under a specific physiological condition.

Furthermore, it is considered that the composition 10 comprises a plurality of formulation agents 12 configured together, in particular either to be degraded differently under the same specific physiological condition, or to be degraded under different physiological conditions. For example, several formulation agents 12 could be arranged in a configuration into successive concentric layers; more particularly, a first formulation agent comprising a first dose of a first hydride could be coated with a second formulation agent different from the first formulation agent and comprising, where appropriate, a second dose of a second hydride, the first and second hydrides could be different from each other and the first and second doses could be different from each other. An example of a configuration alternative to a configuration into successive concentric layers may consist in superimposing substantially planar layers. For example, it could also be considered that inclusions of a first formulation agent 12 comprising a first dose of a first hydride are bonded together by a hydride-free second formulation agent 12.

Whether one single coating 122 (or binder 123) or a plurality of coatings 122 (or binders 123) is used, the embodiment of the invention with the coating 122 (or binder 123) could define a delivery of molecular dihydrogen on the long run, targeted or expanded (for example over the gastrointestinal tract), irrespective of the mode of administration selected in particular amongst an ophthalmic, cutaneous, percutaneous, vesical, intracranial, oral, rectal and vaginal administration.

As it will be described in more details later on with reference to FIG. 3, given the compositions of the formulation agent 12 set out hereinabove, it should be understood that all it needs is to vary the amount or the relative arrangement of the formulation agent 12 or of a set of formulation agents 12 so as to vary the time and location where the hydride will be released in the body of the subject.

Thus, by degradation of the formulation agent 12 and dissolution of the hydride 11, the composition 10 allows releasing dihydrogen in at least one targeted portion of the human or animal body, this targeted portion being defined by one or several specific physiological conditions and/or by said specific external stimulus. Furthermore, by degradation of the formulation agent 12 and dissolution of the hydride 11, the composition 10 allows releasing dihydrogen over a controlled period of time, and where necessary longer than the durations of delivery achieved with the administration methods according to the prior art.

Henceforth, the released hydrogen molecules could be absorbed or assimilated by the organism, and more particularly by the target member or tissue. Thus, the therapeutic qualities of dihydrogen are essentially beneficial to said target member or tissue, the hydrogen molecules being barely delivered, or not at all, somewhere else other than at the level of this target member or tissue.

Hence, the invention provides for storing the hydrogen in a hydride able to dissolve on contact with water. The stored amount of hydrogen that could be released is compatible with the targeted application. Their abundance, their low cost, their capability of releasing a significant mass of dihydrogen (from 1 to 7.6% of released hydrogen with respect to the mass of the product) and their non-toxicity make them ideal candidates. A metallic hydride is composed by metallic atoms which form a host network for hydrogen atoms trapped in interstitial sites, such as the surface of the metal or network defects. A hydride able to dissolve on contact with the water present preferably has, and in particular at its potential contact surface with a surrounding aqueous medium, a significant number of “—H” terminations or functional groups that are able to combine spontaneously with H2O molecules while releasing molecular hydrogen and while forming a passivating oxide layer at the surface of the hydride. The silicon, magnesium and calcium hydrides, in particular non-functionalised, are capable of giving rise to such recombinations.

Furthermore, the hydride 11 may be in different forms.

First of all, the hydride may be porous in particular in order to enlarge the surface of contact of the hydride with the surrounding aqueous medium and thus increase the rate, or equivalently, the flow rate of delivery of dihydrogen.

To obtain high rate and flow rate of delivery of dihydrogen, it is also considered to use a hydride reduced in powder. Preferably, the hydride 11 powder than has particles 111 with an average size comprised between 10 nm and 10 μm. In addition, an association of several hydrides, such as a calcium hydride and/or a titanium hydride and/or a magnesium hydride, and/or an association of several dopants could be considered. For example, a mechanical crushing of magnesium hydride with 20% of calcium hydride for 10 hours enables the creation of defects at the surface of the particles of the hydride 111 and accelerates the hydrolysis rate by 6.

A preferred embodiment of the invention consists in using porous silicon as a hydride 11.

Methods for producing porous silicon, in particular porous silicon that could be reduced into powder, are known under the following denominations:
chemical dissolution (or “stain etching”) which is described in particular in the article of DIMOVA-MALINOVSKA D., SENDO VA-VASSILEVA M., TZENOV N., KAMENOVA M., entitled “Preparation of thin porous silicon layers by stain etching” and published in Thin Solid Films, 1997, 297, pp. 9-12;
plasma etching (or “spark etching”) which is described in particular in the article of HUMMEL R. E., MORRONE A., LUDWIG M., CHANG S.-S., entitled “On the origin of photoluminescence in the spark-eroded silicon” and published in J. Appl. Phys., 1993, 63, pp. 2771-2773; and
electrochemical anodisation which is described in particular in the article of SMITH R. L., COLLINS S. D., entitled “Porous silicon formation mechanisms” and published in J. Appl. Phys., 1992, 71, 8, pp. R1-R7 and the article of LEHMANN V., GOSELE U., entitled “Porous silicon formation: a quantum wire effect” and published in Appl. Phys. Lett., 1991, 58, pp. 856-858.

The first two methods allow making porous silicon layers with a thickness in the range of a few microns. In turn, the electrochemical anodisation allows obtaining thicker layers.

Whether the hydride 11 consists of porous silicon or others, when it is in the form of powder, its particles 111 have an individual or average size comprised between 10 nm and 10 μm. The smaller the size of the particles, the larger will be the amount of embedded hydrogen. For example, one single SiH₄ molecule will release two hydrogen molecules.

It could be desired to have a low rate and flow rate of delivery of dihydrogen, in particular for a prolonged action of the composition 10 over time. In which case, it will be preferably to use a non-porous hydride 11, in a volume form offering a limited contact surface by its shape with the aqueous medium intended to dissolve it.

Regardless of the composition or the form of the hydride 11, the hydrogen stored therein is therefore formulated so as to be dispensed in a targeted manner and at accurate doses using the water present in the human or animal body. Indeed, in a composition as described hereinabove, it is easily possible to finely control the amount of hydride present in the composition. Of course, this amount of hydride is proportional to the amount of dihydrogen that will be delivered to the target member or tissue. In particular, it is possible to calculate the amount of porous silicon, for example of formula SiH₄, equivalent to 1L of saturated hydrogen water, in terms of dihydrogen supply to the human or animal subject. For example, a composition capable of delivering 1% of its mass in the form of dihydrogen should be absorbed on a daily basis in an amount of 157 mg to enable the release of 1.57 mg of dihydrogen. It should be noted that this calculation could also be carried out for a hydride of formula CaH₂ or MgH₂. For example, an association of MgH₂ with TiH₂ allows for a desorption yield of 4.9 weight % namely a daily dose of 30 mg of hydride. Hence, the control of the amount of hydride in the composition 10 allows finely controlling the dose of molecular hydrogen that will be delivered to the subject, and more particularly to the target member or tissue.

It should be understood that, in comparison with the known delivery techniques, all of the dihydrogen delivered thanks to the composition 10 according to the invention will be beneficial essentially, and possibly exclusively, to the target member or tissue, and that being so over a controlled period of time. Once the delivery is targeted in this manner, the amount of dihydrogen delivered thanks to the composition 10 according to the invention could be smaller than the amounts of dihydrogen delivered in a non-targeted manner by the known techniques, while featuring at least as much benefits for the target member or tissue.

The interest lies not only in a “targeted” delivery but also in a remote delivery with i) more deliverable H2, ii) a more stable concentration over time, iii) a lower observance.

Similarly, once the dihydrogen is delivered in a targeted manner thanks to the composition 10 according to the invention, the benefit obtained from this delivery is no longer limited by the half-life of dihydrogen in the human or animal body. Indeed, the dihydrogen acts without delay on the target member or tissue. Hence, the effective dose is reduced in comparison with a dispensing by hydrogen water.

The composition 10 according to the invention also facilitates the observance of the hydrogen therapy the implementation of which is enabled thereby, whether for the patient or for the medical staff. Furthermore, the patient benefits from a psychological effect related to the intake of a medicine, a benefit that he could not, or could barely, enjoy in case of an administration of hydrogen water by ingestion.

Another advantage of the composition 10 according to the invention is that it enables the delivery of dihydrogen, without any delivery of dioxygen.

Other advantages will appear from the description made hereinbelow of different embodiments of the composition 10 and of the associated delivery device 1.

FIGS. 1A to 1E illustrate sectional views of different embodiments of the composition 10 and/or of the targeted delivery device 1 according to the invention. Indeed, according to another aspect, the invention relates to a targeted delivery device 1 comprising a composition 10 as described hereinabove.

FIG. 1A illustrates an embodiment according to which the targeted delivery device 1 is such that it enables a cutaneous administration of the composition 10. The targeted delivery device 1 as illustrated is a plaster 1C comprising the composition 10 kept in contact with the skin through a skin possibly extending in areas configured to adhere to the skin of the subject 2. In the illustrated example, the composition 10 comprises particles 111 of the hydride 11 bonded together by a binder 123 which could be in the form of a gel for example. In this embodiment, the dissolution of the composition 10 could be obtained by humidifying the plaster 1C containing the powder or by using the fluids of the organism, such as sweat in particular. Alternatively or complementarily, it could also be considered to allow wetting the plaster 1C extemporaneously by adding water from outside the human or animal body. This embodiment is particularly suited for the treatment of psoriasis or cutaneous ulcers for example.

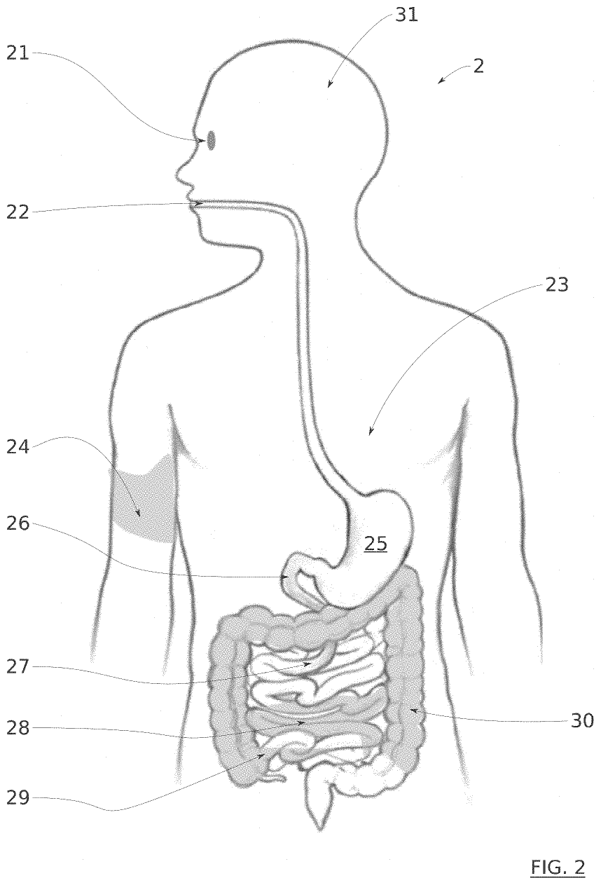

FIG. 1B schematically illustrates an embodiment according to which the targeted delivery device 1 is a pill 1A. The pill 1A may be formulated so as to be administered through an oral, rectal or vaginal route. Preferably, it comprises a coating 122 within which the hydride 11 is kept until the coating 122 is degraded. The hydride 11 may be in at least one amongst any of the forms detailed hereinabove. Referring to FIG. 2, this embodiment is particularly suited for the delivery of molecular hydrogen at one or several specific location(s) of the gastrointestinal tract: the mouth 22 (for any treatment of the periodontium for example), the oesophagus and the stomach 25 (for the treatment of the ulcer for example), but also the small intestine for chronic inflammatory bowel disease (IBD) (duodenum 27, jejunum 28, ileum 29), or the colon 30, using a gastro-resistant coating 122 that could dissolve at different pH. In this embodiment, the dissolution of the composition 10 could be obtained by humidifying the pill 1A using the fluids found on the gastrointestinal tract.

According to one embodiment, the molecular hydrogen delivered by the dissolution of the composition 10 could advantageously be detected by an internal, mobile or implanted sensor, preferably disposed in the stomach 25 of the human or animal body 2. For example, a mobile stomach dihydrogen sensor, such as the sensor known from the document WO 2018/02031 A1, is then ingested concomitantly with the pill 1A and comprises a wireless transmission device to transmit a signal outwardly of the body when a dihydrogen capture measurement is recognised. According to another example, said sensor could also be joined with device implanted preferably at the wall of the stomach, such as the device known from the document FR 3059558 A1 which also comprises a wireless transmission device for the aforementioned purposes. More particularly, each wireless transmission device is capable of transmitting a signal to a wireless reception device, external to the human or animal subject, to record and check up the act of ingesting the pill 1A. It should then be understood that the dihydrogen released by the hydride 11 has both a therapeutic role and a detection agent 13 role enabling measurement of the therapeutic observance.

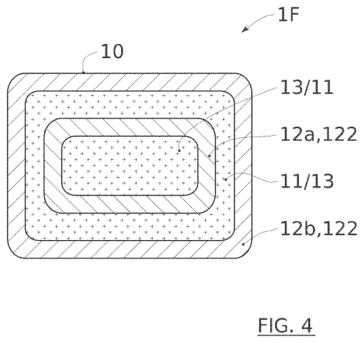

According to one variant, and example of which is illustrated in FIG. 4, it could be considered that one pill 1F comprises several layers that differ by their composition and specific relative arrangement according to the portion of the gastrointestinal tract that is treated by delivery of molecular hydrogen. Thus, referring to FIG. 4, the pill 1F may comprise for example at least two successive concentric layers of one or several formulation agents 12 each comprising a container 121, a coating 122 and/or a binder 123, wherein the hydride 11 is held until the container 121, the coating 122 and/or the binder 123 is degraded. An external first layer 12b could be degraded at the level of the mouth 22 or the oesophagus of the human or animal subject 2 for treatment thereof by the molecular hydrogen. An internal second layer 12a, for example whose coating 122 is degraded at a low pH is degraded afterwards preferably in the stomach 25, thereby inducing the release of molecular hydrogen for measuring the therapeutic observance. It could also be considered that the external first layer 12b is preferably degraded in the stomach 25 of the human or animal subject 2, thereby inducing the release of molecular hydrogen for measuring the therapeutic observance; and that an internal second layer 12a is degraded afterwards in a downstream portion of the gastrointestinal tract, such as the small intestine (duodenum 27, jejunum 28, ileum 29), the intestine or the colon 30, using a gastro-resistant coating 122 that could dissolve at different pH, to release the molecular hydrogen therein for therapeutic purposes.

Different combinations of these particular embodiments could also be considered. For example, the pill 1F may comprise at least three successive concentric layers able to deliver molecular hydrogen in different portions of the gastrointestinal tract and for therapeutic and/or observance measurement purposes.

According to another example, the detection agent 13 may consist of a fluorophore that could be detected by an optical imaging device external to the patient.

FIG. 1C schematically illustrates an embodiment according to which the targeted delivery device 1 is an implant 1E. Referring to FIG. 2, this implant could be in any form that is suited to the location, such as an arm 24, the heart 25, a brain ventricle 31, of the human or animal body where it should be implanted for the subject 2 to benefit from. For example, the implant 1E may comprise the hydride in a volume form or in the form of powder whose particles are bonded together by a binder 123. It could also be considered that the composition 10 comprises a coating 122, as a formulation agent 22, suited specifically to the instrument used to carry out the implantation and to the implantation operation itself. In this embodiment, the dissolution of the composition 10 may be obtained by humidifying the implant 1E using the fluids present at the location of the implantation. In this embodiment, the delivery is preferably controlled with a delayed and prolonged effect by encapsulation or coating of the hydride 11 in a PLA (“polylactic acid”) type biodegradable polymer, as a formulation agent 12. More particularly, it could be considered that the implant comprises several layers that are different by their composition and specific relative arrangement so as to be biodegradable over a week-long or month-long period of time, the different layers containing the hydride 11 that dissolves in the presence of the extracellular fluids of the physiological environment where the implantation is carried out. For example, such an implant with several coating layers 122 may comprise a PLA-PEG (polyethylene glycol)-PLA type stacking. In this embodiment, the use of silicone could also be considered in particular to vary the availability of the hydride 11 in the human or animal body 2. In particular, thanks to the invention according to this embodiment, it could be considered to carry out the delivery of molecular hydrogen also by diffusion activated by an external energy source, for example the aforementioned ultrasounds, or by puncturing in members, for example by puncturing the myocardium, in particular in an acute treatment of the myocardial infarction, in particular in combination with the treatment by introduction of strain cells, or by release into the brain ventricles. Thanks to the invention according to this embodiment, it could also be considered that the hydride 11 wraps at least partially a medical device 40 or an element 41 of an implantable medical device, such as a pacemaker or a stimulation electrode or a probe. Preferably, the coating 122 is then biodegradable and contains the hydride that could then be released upon the implantation or in a differed manner by degradation of the coating or internal or external stimulation. The medical device 40 could be a pacemaker, a stent, a biliary or endovascular prosthesis, or any device generating an inflammation upon implantation thereof. For example, the stimulation electrode could be a deep stimulation electrode implanted into the brain 31 of the patient, in particular to treat Parkinson's disease. In particular, the dihydrogen released in this manner in a dissolved form could allow fighting possible inflammation phenomena related to the implantation. It is also possible to consider an administration by catheterisation, for example intravesical, by coating the probe.

FIG. 1D schematically illustrates an embodiment according to which the targeted delivery device 1 is a contact lens, potentially a corrective one. The face intended to be in contact with the eye 21 of the subject 2 (Cf. FIG. 2) may be covered at least partially with the composition 10 according to the invention. In this case, the composition comprises, for example, an ophthalmic gel bonding particles 111 of the hydride 11. Alternatively or complementarily, the lens could be made based on a non-biodegradable and/or non water-miscible polymer, such as a silicone; inclusions of particles 111 of the hydride 11 could line the face of the lens intended to be in contact with the eye 21. Delivery to the eye 21 is particularly suited for the treatment of retinopathies or of an optic nerve crush. In this embodiment, the dissolution of the composition 10 could be obtained by humidifying the composition 10 using the lacrimal liquid hat flows at the outer surface of the eye 21.

FIG. 1E schematically illustrates an embodiment according to which the targeted delivery device 1 is a capsule 1B. The capsule 1B may be formulated so as to be orally administered. Complementarily, it could have the shape and the outer composition of a suppository and be administered through a rectal or vaginal route. Preferably, it comprises a container 121 in which the hydride 11 is held until the container 121 is degraded. The hydride 11 may be in at least one of the forms detailed hereinabove. Referring to FIG. 2, this embodiment is particularly suited for the delivery of molecular hydrogen at one or several specific location(s) of the gastrointestinal tract: the mouth 22 (for any treatment of the periodontium for example), the oesophagus and the stomach 25 (for the treatment of the ulcer for example), but also the small intestine (duodenum 26, 27, jejunum 28, ileum 29), or the colon 30, using a gastro-resistant container 121 that could dissolve at different pH. In this embodiment, the dissolution of the composition 10 could be obtained by humidifying the capsule 1B using the fluids found on the gastrointestinal tract. For example, the ingestible capsule 1B could be soluble in the stomach for a stomach release of the molecular hydrogen.

According to another embodiment, the targeted delivery device 1 is a liquid or a past that could pass through a needle or a catheter and containing the hydride in a nanoparticle form in a biodegradable coating. This formulation enables administration by injection.

FIG. 3 schematically illustrates the adaptation that could be done with the targeted delivery device 1 according to the embodiment of the invention in the form of a capsule 1B. The abscissa X-axis illustrates changes in the composition, or equivalently in the nature, herein three in number, of the encapsulant 121, whereas the ordinate E-axis illustrates an increase in the thickness of the encapsulant 121. Thus, this figure illustrates the adaptation capacity offered by the capsule 1B according to the invention to ensure a targeted delivery of dihydrogen at different locations 22, 25 to 30 (Cf. FIG. 2) of the human or animal body 2. The point of delivery of the hydride 11 could be programmed by the thickness of the gastro-resistant polymer which determines the duration of the dissolution of the capsule 1B. With a small thickness of the film of the capsule 1B, namely a small amount of polymer, a quick dissolution is achieved and therefore potentially a delivery at the level of the anterior intestinal tract 27, and therefore at the duodenal level. Conversely, a large polymer thickness defers the complete dissolution of the capsule 1B and promotes a jejunal 28, and possibly ileal 29 or colic 30, delivery, depending on the targeted location (Cf. FIG. 2). Similarly, the hydride 11 could be mixed with an excipient, serving as a binder 123, to form a pill 1A or a tablet that will dissolve substantially slowly or quickly. The ingestible pill 1A or capsule 1B could be soluble in the stomach 25 for a stomach release of hydrogen. Thus, the adaptation as illustrated in FIG. 3 is deemed to be easily transposable to the embodiments of the invention other than that illustrated in this figure.

According to one example, the composition 10 comprises at least one hydride 11, this hydride being a silicon hydride. The dissolution of a silicon hydride 11 allows limiting, and even avoiding, a modification of the pH of the solution in which it is dissolved, and more particularly an increase of this pH. Thus, a composition 10 comprising a silicon hydride 11, and possibly only silicon hydride 11 without any other ion hydride, is particularly suited for the delivery of dihydrogen in pH-sensitive media, in particular in the human or animal body. As examples of pH-sensitive media, mention may be made of:
the vagina, typically having a pH comprised between 3.8 and 4.5,
the skin, typically having a pH comprised between 4 and 6, and in particular comprised between 4 and 5,
the eye, for which the teardrops typically have a pH of substantially 7.4,
the internal medium, typically having a pH comprised between 7.35 and 7.45.

Moreover, the rate of dissolution of the ion hydrides 11, for example of the magnesium hydride 11, and that of the silicon hydride 11 differ from each other according to the pH of the environment of the composition 10. Hence, the release of dihydrogen differs between these hydrides 11 according to the pH. A silicon hydride 11 has a slow rate of dissolution, and even no dissolution, at an acid pH, for example lower than 7.4, in comparison with a magnesium hydride 11. A silicon hydride 11 has a quicker rate of dissolution at a basic pH, for example higher than 7.4, in comparison with the rate of dissolution of a magnesium hydride 11. At a substantially neutral pH, for example of about 7.4, the silicon hydride 11 and the magnesium hydride 11 could dissolve by releasing a similar amount of dihydrogen, the silicon hydride 11 further having the advantage of not modifying the pH of the solution in which the composition 10 is dissolved.

Upon the dissolution of the silicon hydride 11 in a basic pH, in particular higher than 7.4, the reaction of the hydrogen atoms could be illustrated by the following reaction (I), inducing the release of dihydrogen.

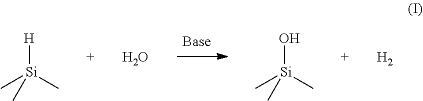

Upon the dissolution of the silicon hydride in a basic pH, an oxidation of the bonds between the silicon atoms also takes place and could be illustrated by the following reaction (II), inducing the release of dihydrogen.

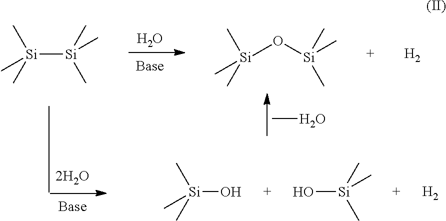

Hence, the release of dihydrogen originating from a silicon hydride is promoted in a basic medium and does not induce the production of additional hydroxide ions, as is the case for ion hydrides. Indeed, for ion hydrides, for example calcium hydride or magnesium hydride, the hydride ions H⁻ react with water to form dihydrogen and hydroxide ions. More particularly, the hydride ions H⁻ react with water to form hydroxide compounds, for example Ca(OH)₂ or Mg(OH)₂, and dihydrogen. Afterwards, these hydroxide compounds are hydrolysed, thereby causing the dissociation of the metallic ions, for example Ca²⁺ or Mg²⁺, and hydroxide ions.

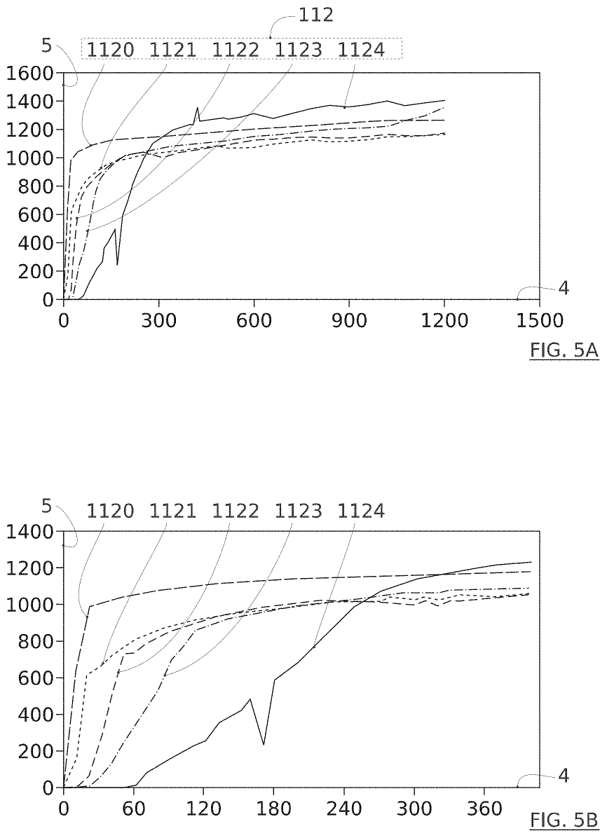

For example, FIGS. 5A and 5B illustrate the kinetics 112 of dihydrogen release 5 in μg/L, as a function of time 4 in seconds, induced by the degradation of a composition 10 comprising substantially 13 mg of magnesium hydride, in 30 mL of a Phosphate Buffer Salin PBS solution:
1120: the PBS solution having a pH 5.87,
1121: the PBS solution having a pH 6.5,
1122: the PBS solution having a pH 6.95,
1123: the PBS solution having a pH 7.4,
1124: the PBS solution having a pH 8.45.

As illustrated in FIGS. 5A and 5B, the dissolution of the magnesium hydride occurs preferably at an acid pH. The required time for reaching the release of about 1 ppm of dihydrogen, after immersion of this composition 10, is about 30 seconds at pH 5.8, 4 minutes at pH 7.4 and 5 minutes at pH 8.45. Furthermore, it has been measured that the pH of the PBS solution becomes basic, and rises up to pH 10, and possibly 11, following the dissolution of the magnesium hydride.

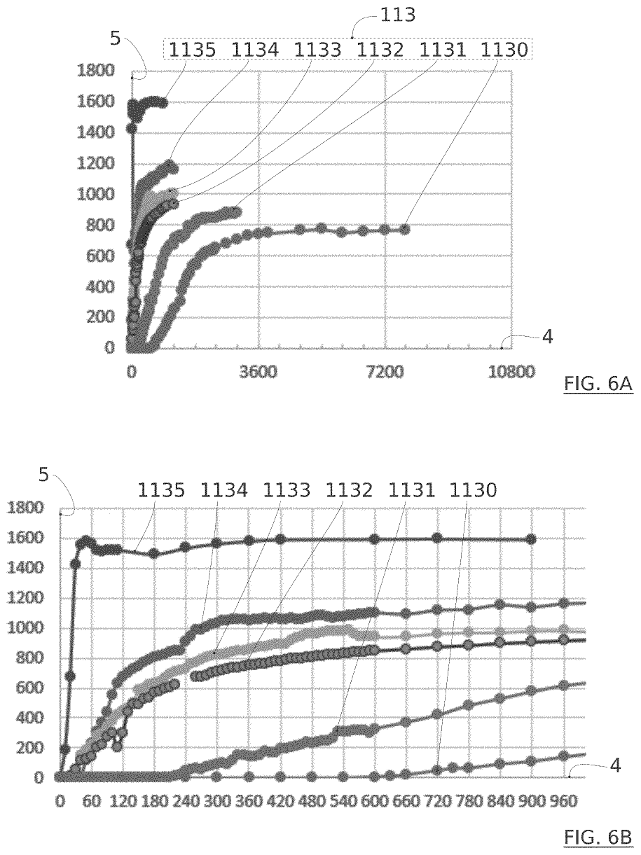

For example, FIGS. 6A and 6B illustrates the kinetics 113 of dihydrogen release 5 in μg/L, as a function of time 4 in seconds, induced by the degradation of a composition 10 comprising substantially 13 mg of silicon hydride, in 30 mL of a Phosphate Buffer Salin PBS solution:
1130: the PBS solution having a pH 5.8,
1131: the PBS solution having a pH 6.48,
1132: the PBS solution having a pH 6.95,
1133: the PBS solution having a pH 7.3,
1134: the PBS solution having a pH 8.48,
1135: the PBS solution having a pH 11.

As illustrated in FIGS. 6A and 6B, no release of dihydrogen is observed in the first 15 minutes at pH 5.8. Complementary tests have shown that no release of dihydrogen is observed at pH 3, a pH equivalent to that of the stomach, neither at pH 4.2, a pH equivalent to that of the vagina, for at least 4 hours. Hence, the kinetics of release of dihydrogen could be much slower for a silicon hydride in comparison with a magnesium hydride, in particular for a pH lower than 7.4, and possibly a pH lower than 6. Furthermore, it has been measured that the pH of the PBS solution remains substantially equal to the initial pH following the dissolution of the silicon hydride. For a pH lower than 7.4, and possibly a pH lower than 6, the dissolution of the silicon hydride allows maintaining the pH of the solution in which it is dissolved, and a prolonged release of dihydrogen over time, for example over a duration longer than 5 hours, and possibly longer than 10 hours.

Thus, it should be understood that a composition 10 comprising a silicon hydride 11, and possibly only silicon hydride 11 without any other ion hydride, could release no dihydrogen in the stomach and would release dihydrogen at a basic pH, for example in the intestine. The composition 10 could then be free of any gastro-resistant formulation agent 12, and more particularly of any coating based on a gastro-resistant material, while enabling a targeted delivery of dihydrogen in an environment at a basic pH such as the intestine. A composition 10 comprising a silicon hydride 11, and possibly only silicon hydride 11 without any other ion hydride, could cause a slow release of dihydrogen at a neutral pH, for example equal to about 7.4, or slightly basic, for example comprised between 7.4 and 8. A slow release of dihydrogen is particularly advantageous when the composition is associated to an implant, to fight possible inflammation phenomena related to the implantation on the long-run, and in particular over a longer duration than with an ion hydride.

The composition 10 may comprise a mixture of hydrides 11, as set out before. In particular, the kinetics of release of release of dihydrogen in an acid medium, for example for a pH lower than 7.4, and possibly lower than 6, could be accelerated. More particularly, the composition 10 may comprise a mixture of hydrides 11 comprising a silicon hydride 11 and at least one other hydride 11, in particular an ion hydride such as calcium hydride or magnesium hydride. Thus, the dissolution of the ion hydride 11 could cause a release of dihydrogen and the production of hydroxide ions. The hydroxide ions could then react with the silicon hydride 11 to induce the release of dihydrogen. The properties of a silicon hydride 11 and of an ion hydride 11 could then be used synergistically to induce a release of dihydrogen, in particular in an acid medium. Thus, a high hydrogen release yield could be obtained. A composition 10 comprising a mixture of hydrides 11 comprising a silicon hydride and at least one other hydride is thus particularly suited for a use, for example in acid compartments of the human body such as the vagina, the skin, and the stomach, and possibly slightly basic ones such as the internal medium and the intestine.

Depending on the relative proportion between the silicon hydride and the at least one other hydride in the mixture of the composition 10, the kinetics of release of dihydrogen could be modulated so as to be more or less quick upon dissolution of the mixture.

According to one example, the composition 10 comprises a mixture of hydrides 11, comprising a silicon hydride and at least one other hydride, the proportion of silicon hydride being higher than 20 weight %, and possibly higher than 50 weight %, and possibly higher than 75 weight % with respect to the total mass of hydride.

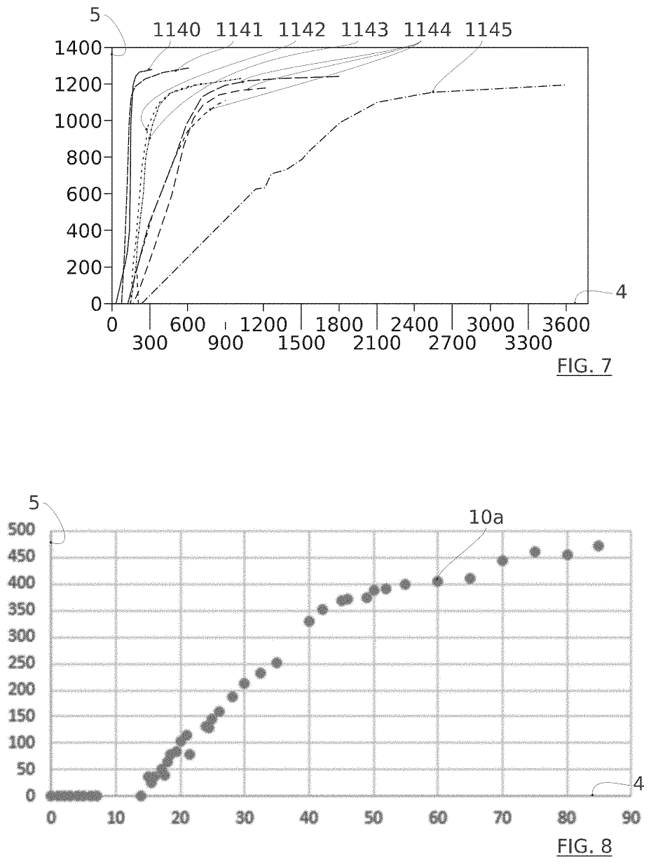

For example, FIG. 7 illustrates the kinetics of release of dihydrogen 5 in ppb (abbreviation of part per billions), as a function of time 4 in seconds, induced by the dissolution of the following compositions:
1140: a composition 10 comprising 7.2 mg of magnesium hydride,
1141: a composition 10 comprising 5.5 mg of magnesium hydride and 1.7 of silicon hydride, namely about 24 weight % of silicon hydride with respect to the total mass of hydride,
1142: a composition 10 comprising 1.1 mg of magnesium hydride and 6.1 of silicon hydride, namely about 85 weight % of silicon hydride with respect to the total mass of hydride,
1143: a composition 10 comprising 1.7 mg of magnesium hydride and 5.5 of silicon hydride, namely about 76 weight % of silicon hydride with respect to the total mass of hydride,
1144: three compositions 10 comprising
   0.3 mg of magnesium hydride and 6.9 of silicon hydride, namely about 96 weight % of silicon hydride with respect to the total mass of hydride,
   0.4 mg of magnesium hydride and 6.8 of silicon hydride, namely about 94 weight % of silicon hydride with respect to the total mass of hydride,
   0.6 mg of magnesium hydride and 6.6 of silicon hydride, namely about 92 weight % of silicon hydride with respect to the total mass of hydride,
1145: a composition 10 comprising 7.2 mg of silicon hydride.

As illustrated in FIG. 7, it is actually observed that the kinetics of release of dihydrogen could be modulated so as to be more or less quick upon the dissolution of the mixture, according to the relative proportion between the silicon hydride and the at least one other hydride in the mixture of the composition 10. The larger the proportion of silicon hydride, the more the kinetics of release of dihydrogen would tend towards that induced by the dissolution of a silicon hydride alone.

It has been demonstrated that a silicon hydride could dissolve slowly, and even not at all, at an acid pH, for example at a pH lower than 7.4. The silicon hydride could be formulated in a formulation agent 12 comprising a solution at a pH lower than 7.4, and possibly a pH lower than 6. Thus, the composition 10 could be preserved, while limiting and even avoiding the degradation of the hydride. When the composition is disposed in an environment with a substantially neutral or basic pH, having a pH higher than or equal to 7.4, a formulation agent 12 could be configured so as to be degraded and/or be semi-permeable to an element of the environment to cause contact of the silicon hydride 11 with this element, and thus induce the dissolution thereof and the release of dihydrogen.

The formulation agent 12 could be semi-permeable to water, to ions and to gases so as to isolate the hydride 11 from the environment of the composition 10, while enabling the release of dihydrogen. Thus, the composition 10 could be particularly suited for the delivery of dihydrogen in the eye, by avoiding the dispersal of the hydride, for example in the form of powder. The composition 10 could be in the form of a contact lens. For example, the formulation agent 12 could be a hydrogel, and in particular a hydrogel that is semi-permeable to water, to ions and to gases.

For example, a composition 10 comprising a poly(vinyl alcohol), abbreviated PVA, gel, rinsed with an acid buffer, and comprising a silicon hydride. FIG. 8 is a graph of the kinetics of release of dihydrogen 5 in ppb, as a function of time 4 in minutes, induced by the dissolution of the composition 10a according to this example. Once the composition 10a is placed in a solution at pH 7.4, a release of dihydrogen at a slow rate is observed. For 7 mg of silicon hydride in 1.4 mL of a PVA gel, a release of 400 ppb of dihydrogen is obtained after 1 hour in a beaker of 100 mL of PBS buffer at pH 7.4.

The present invention finds a particularly advantageous application in the therapeutic treatment of any disease, including in particular the 166 pathologies that are inventoried in the articles mentioned in the introduction of the present application. In particular, its use in the treatment of at least one cardiovascular disease, such as the myocardial infarction, or in the treatment of at least one neurodegenerative disease, such as Parkinson's disease and Alzheimer's disease, could be considered.

The invention is not limited to the previously-described embodiments and encompasses all embodiments covered by the claims.

1. A composition comprising:
a hydride that will dissolve on contact with an aqueous medium, thereby releasing dihydrogen as a solute into the aqueous medium, and
a formulation agent configured to bring the hydride into contact with the aqueous medium found in a human or a non-human animal body.

2. The composition according to claim 1, wherein the formulation agent is selected from the group consisting of a container, a coating applied to the hydride, a coating applied to particles of the hydride, a binder holding an agglomeration of particles of the hydride, and combinations of thereof.

3. The composition according to claim 1, wherein the hydride is porous.

4. The composition according to claim 1, wherein the hydride is in the form of powder whose particles have an average size of between 10 nm and 10 μm.

5. The composition according to claim 1, wherein the hydride is selected from the group consisting of silicon hydride, magnesium hydride, calcium hydride, and mixtures thereof.

6. The composition according to claim 1, wherein the hydride comprises porous silicon.

7. The composition according to claim 1, wherein the formulation agent is selected from the group consisting of
gastro-resistant materials,
materials soluble on contact with the aqueous medium having a pH range associated with a predetermined administration site on the human or non-human animal body,
biodegradable polymers,
materials that will degrade in response to a predetermined level of external stimulation
materials soluble on contact with the aqueous medium,
gels, and
combinations thereof.

8. The composition according to claim 1, wherein the formulation agent will degrade in response to a sufficient level of an external or an internal stimulus.

9. The composition according to claim 1, wherein the formulation agent has a determined degradation rate in the aqueous medium found at a predetermined site on the human or non-human animal body.

10. The composition according to claim 1, wherein the formulation agent is semi-permeable.

11. The composition according to claim 1, comprising a plurality of formulation agents configured together, in successive concentric layers or in superimposed planar layers, so as to be degraded either differently on contact with the aqueous medium found at a predetermined site on the human or non-human animal body, or on contact with the aqueous medium found at different predetermined site on the human or non-human animal body.

12. The composition according to claim 1, further comprising:
at least one detection agent by which ingestion of the composition can be confirmed.

13. A medicant composition comprising:
a hydride that will dissolve on contact with an aqueous medium thereby releasing dihydrogen as a solute into the aqueous medium, and
at least one formulation agent configured to bring the hydride into contact with the aqueous medium found in a human or a non-human animal body.

14. A therapeutic composition for the treatment a cardiovascular or neurodegenerative disease, the composition comprising:
a hydride that will dissolve on contact with an aqueous medium thereby releasing dihydrogen as a solute into the aqueous medium, and
at least one formulation agent configured to bring the hydride into contact with the aqueous medium found in a human or a non-human animal body.

15. A device for targeted delivery of dihydrogen, in a human or non-human animal body, the device comprising a composition consisting of:
a hydride that will dissolve on contact with an aqueous medium thereby releasing dihydrogen as a solute into the aqueous medium, and
at least one formulation agent configured to bring the hydride into contact with the aqueous medium found in a human or a non-human animal body.

16. The device according to claim 15 formulated for use with at least one administration route selected from the group consisting of oral, parenteral, rectal, vaginal, ophthalmic, cutaneous, transdermal, and respiratory administration.

17. The device according to claim 15, configured as a pill, a capsule, a plaster, a contact lens, or an implant.

18. The composition according to claim 1, formulated so as to be adapted to at least one administration route selected amongst an oral, parenteral, rectal, vaginal, ophthalmic, cutaneous, transdermal and respiratory administration.

19. The composition according to claim 1, formulated so as to be administered in one of the following forms: a pill, a capsule, a plaster, a contact lens, or an implant.

20. A method for releasing hydrogen molecules in the human or non-human animal body using a composition according to claim 1.
